SEQUENCE LISTING (13 sequences)

SEQ ID NO: 1 - DNA, 234 bp, Sorghum bicolor
ggtgccgcaggagccgttcctgttcgcggcgagcatccacgacaacatcgcgtacgggcgcgagggcgcgacggaggcggaggtggtggaggcggcgacgcaggcgaacgcgcaccggttcatctcggcgctgccggagggctacgggacgcaggtgggcgagcgcggggtgcagctgtcgggcgggcagcggcagcggatcgcgatcgcgcgcgcgctggtgaagcaggcggc

SEQ ID NO: 2 - DNA, 228 bp, Sorghum bicolor
ggtgccgcaggagccgttcctgttcgcggcgagcatccacgacaacatcgcgtacgggcgcgagggcgcgacggaggcggaggtggtggaggcggcgacgcaggcgaacgcgcaccggttcatctcggcgctgccggagggctacgggacgcaggtgggcgagcgcggggtgcagctgtcgggcgggcagcggatcgcgatcgcgcgcgcgctggtgaagcaggcggc

SEQ ID NO: 3 - DNA, 22 bp, Sorghum bicolor
ctcctcgccgtcttcccgctcg

SEQ ID NO: 4 - DNA, 22 bp, Sorghum bicolor
gcggcaccgcctgtcagccgct

SEQ ID NO: 5 - DNA, 26 bp, Sorghum bicolor
cgttcaacgcggagcgcaagatcacg

SEQ ID NO: 6 - DNA, 22 bp, Sorghum bicolor
cgcctgtcagccgctgcagctg

SEQ ID NO: 7 - DNA, 21 bp, Sorghum bicolor
gctcttcgaggccaaccttcg

SEQ ID NO: 8 - DNA, 22 bp, Sorghum bicolor
cgtcgatcacggcgatggtgtg

SEQ ID NO: 9 - DNA, 26 bp, Sorghum bicolor
cgtcctgcagaagatgttcatgaagg

SEQ ID NO: 10 - DNA, 23 bp, Sorghum bicolor
gtgcgccaccacgatggtggtgc

SEQ ID NO: 11 - DNA, 20 bp, Sorghum bicolor
cgaggccgtcgccaacctgc

SEQ ID NO: 12 - DNA, 21 bp, Sorghum bicolor
gagtcggagcggtggctcttc

SEQ ID NO: 13 - DNA, 22 bp, Sorghum bicolor
gttgtacttgcgcacgtccttg

(19) United States
(12) Patent Application Publication
(10) Pub. No.: US 2011/0225665 A1
(43) Pub. Date: Sep. 15, 2011

(54) Stable dw3 Allele for Sorghum and a Molecular Marker to Facilitate Selection

(75) Inventors: Mitchell Reed Tuinstra, West Lafayette, IN (US); Gurmukh S. Johal, West Lafayette, IN (US)

(73) Assignee: Purdue Research Foundation, West Lafayette, IN (US)

(21) Appl. No.: 13/047,594

(22) Filed: Mar. 14, 2011

Related U.S. Application Data
(60) Provisional application No. 61/313,280, filed on Mar. 12, 2010.
(60) Provisional application No. 61/366,430, filed on Jul. 21, 2010.

(51) Int. Cl. A01H 1/00; C07H 21/04; A01H 5/00; A01H 5/10; C12Q 1/68; C07H 21/00

(52) U.S. Cl. 800/260; 536/23.6; 800/320; 800/300; 800/301; 800/302; 435/6.12; 435/6.1; 536/23.1

(57) ABSTRACT

The identification of a stable dw₃ allele and development of molecular DNA markers to facilitate selection of this allele in development of new cultivars provides a simple genetic solution to the problem of tall height mutants in commercial sorghum [Sorghum bicolor (L.) Moench] parent lines and hybrids.

This application claims priority to U.S. provisional patent application No. 61/313,280, filed Mar. 12, 2010 by Mitchell R. Tuinstra and Gurmukh Johal, entitled “A stable dw₃ allele for sorghum and a molecular marker to facilitate selection.” It also claims priority to U.S. provisional patent application No. 61/366,430, filed Jul. 21, 2010 by Mitchell R. Tuinstra and Gurmukh Johal, entitled “A stable dw₃ allele for sorghum and a molecular marker to facilitate selection.”

### STATEMENT CONCERNING FEDERALLY SPONSORED RESEARCH

This invention was made with government support under Prime Award EPP-A-00-06-00016-00, CFDA 98.001, awarded by the U.S. Agency for International Development (USAID) for the project titled, “Sorghum, Millet and Other Grains Collaborative Research Support Program”, also referred to as INTSORMIL. The government has certain rights in the invention.

### FIELD OF THE INVENTION

The identification of a stable dw₃ allele and development of a molecular DNA marker system to facilitate selection of this allele in development of new cultivars provides a simple genetic solution to the problem of height mutants in commercial sorghum [Sorghum bicolor (L.) Moench] parent lines and hybrids.

### BACKGROUND

Sorghum plant height is a quantitative trait controlled by four major genes (Dw₁: Dw₂: Dw₃: Dw₄). Nearly all of the grain sorghum grown in the developed world is produced using semi-dwarf cultivars. These semi-dwarf cultivars commonly are called “3-dwarf” sorghums since they utilize recessive dwarfing alleles at three of the four major plant height genes (dw₁: Dw₂: dw₃: dw₄).

Dw₃ is the only height gene that has been cloned in sorghum. The wild-type allele for Dw₃ encodes an auxin efflux transporter involved in stein internode elongation. The recessive allele of this gene (dw₃) is used to reduce plant height in nearly all commercial grain sorghum cultivars. This allele was originally identified and characterized by Karper (1932). Karper noted that the dw₃ mutation produced a useful dwarf phenotype, but also noted that the dw₃ allele was unstable and reverted to wildtype Dw₃ at a frequency of approximately 1 in 600 plants. These revertant plants are generally termed “height mutants” (FIG. 1). Since commercial sorghum hybrids often are grown at plant populations of 20,000 to 80,000 plants acre⁻¹, can expect approximately 33 to 133 height mutants per acre depending on the genetic background of the hybrid.

After the Dw₃ gene was cloned and its DNA sequence decoded, comparisons of the mutant and wild-type alleles indicated that the recessive dw₃ allele does not produce a functional protein due to a direct intragenic duplication of 882 base pairs in exon 5. Comparisons of DNA sequences of dw₃ with the reverted Dw₃ allele in height mutants demonstrated that the instability of the dw₃ locus was the result of unequal crossing-over between the tandemly duplicated regions that produced one wild-type allele and another allele with 3 tandem repeats.

Farmers dislike height mutants because these off-types are unsightly in commercial grain production fields. Height mutants also cause problems in commercial seed production. Seed producers do not like height mutants because of the effort and cost required to rogue these plants from seed production fields. The commercial seed sector spends millions of US dollars each year managing height mutants in seed production. The development of a genetic solution to this problem would dramatically reduce the “cost-of-goods” of commercial seed; thereby improving profitability. Moreover, companies that develop “height-mutant free” hybrids will have a strong competitive advantage in the market place.

### SUMMARY

The identification of a stable dw₃ allele and development of a molecular DNA marker system to facilitate selection for this allele in plant breeding programs provides a valuable tool for addressing the problem of height mutants in sorghum.

A PCR assay was developed to screen for new dw3 alleles. Using this assay, a natural variant of dw₃ was identified, where the mutation and dwarf phenotype were found to be the result of a 6 base pair deletion in the dw₃ gene. Because this mutation is a deletion and not a tandem duplication, the allele is not able to spontaneously revert to Dw₃. This new allele represents a solution to the problem of height mutants in sorghum and has been termed dw₃ₛ for “dw₃ stable”. Plants with the stable dw₃ₛ, allele cannot be differentiated from plants with the unstable dw₃ allele by visual inspection of individual plants. Therefore, allele-specific PCR markers have been developed for the gene to facilitate marker assisted introgression of this allele into elite parent lines. These markers provide an invaluable tool for use in breeding by allowing rapid conversion of elite inbred parent lines for the stable dwarf trait through marker assisted introgression.

An isolated nucleic acid molecule includes a fragment of the nucleotide sequence of the sorghum dw₃ gene, wherein the fragment includes a deletion mutation in exon 5 of the dw₃ gene. The deletion mutation is present in a region represented by nucleic acid position 259 to 264 of the exon 5 and includes absence of contiguous nucleic acids GTCGCC in exon 5 of the dw₃ gene.

An isolated nucleic acid molecule includes the nucleotide sequence of the sorghum dw₃ gene, wherein the nucleic acid molecule includes a deletion mutation in exon 5 of the dw₃ gene, designated dw₃ stable (dw₃ₛ). The fragment was amplified by a modified polymerase chain reaction with oligonucleotide primers having nucleotide sequences CGT CCT GCA GAA GAT GTT CAT GAA GG (forward) (SEQ ID NO: 9) and GTG CGC CAC CAC GAT GGT GGT GC (reverse) (SEQ ID NO: 10).

An introgressed sorghum plant includes the nucleic acid responsible for producing a dwarf variety of the sorghum plant. The plant may also be herbicide tolerant, and/or resistant to insects and/or pathogens. The introgressed sorghum plant of may be introgressed with a pollen parent that includes the dw₃ₛ mutant allele.

A hybrid sorghum plant includes the dw₃ₛ gene, wherein the sorghum plant is a dwarf variety. The plant may be introgressed.

A sorghum seed are obtained from an introgressed sorghum plant that includes the dw₃ₛ nucleic acid or from a hybrid sorghum plant.

A plurality of sorghum seeds are obtained from an introgressed sorghum plant or from a hybrid sorghum plant including the dw₃ₛ nucleic acid. The resulting sorghum plants after germination are of uniform height, and do not display any height mutant. The resulting sorghum plants are genetically stable for the dw₃ₛ mutant allele.

A method of producing sorghum plants that have uniform plant height, includes crossing a sorghum plant having the dw₃ₛ nucleic acid with a parental sorghum line to produce sorghum plants having uniform plant height. The resulting sorghum plants may be introgressed, e.g. with one or more hybrid parental lines.

A method of screening for the presence of a deletion mutant in exon 5 of the sorghum dw₃ gene, includes detecting the presence of the deletion mutation in the exon 5 of the sorghum dw₃ gene by a polymerase chain reaction (PCR) performed with oligonucleotide primers having nucleotide sequences CGT CCT GCA GAA GAT GTT CAT GAA GG (forward) (SEQ ID NO: 9) and GTG CGC CAC CAC GAT GGT GGT GC (reverse) (SEQ ID NO: 10). The PCR results in an amplified product of length of about 1071 bp.

A method of screening for the presence of a deletion mutant in exon 5 of the sorghum dw₃ gene, includes detecting the presence of the deletion mutation in the exon 5 of the sorghum by a dw₃ gene sequencing reaction.

Biomarkers for determining the presence of a deletion mutant in exon 5 of the sorghum dw₃ gene include an amplified fragment including the mutation in the exon 5 of the sorghum dw₃ gene. The amplified fragment includes a deletion of contiguous nucleic acids GTCGCC in exon 5 of the dw₃ gene.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. is a photograph of tall dw₃ revertants commonly referred to as “height mutants” showing up in a commercial sorghum production field.

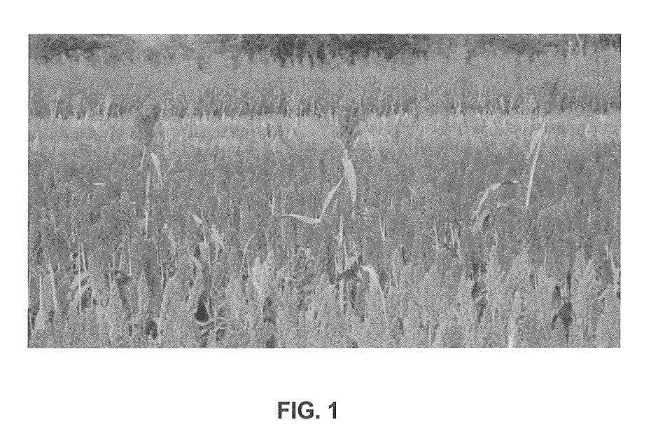

FIG. 2. shows duplicate PCR reactions for El Mota (Dw3), Tx430 (dw₃), and Donor (dw3s) showing the absence of the duplication in El Mota and Donor and the presence of the duplication in Tx430.

FIG. 3, shows sequence alignments of a region of exon 5 of the Dw₃ allele from El Mota (SEQ ID NO: 1) and the dw₃ₛ allele of Donor (Seq. No. 2) showing a 6 base pair deletion that is responsible for the stable dwarf phenotype of the dw₃ₛ allele.

The sequence descriptions and Sequence Listing attached hereto comply with the rules governing nucleotide sequence disclosures in patent applications as set forth in 37 C.F.R. §1.821-1.825.

SEQ ID NO: 1 Dw₃ Exon 5 Region

SEQ ID NO: 2 Dw₃ₛ Exon 5 Region

SEQ ID NO: 3 Dw3/IF Primer

SEQ ID NO: 4 Dw3/IR Primer

SEQ ID NO: 5 Dw3/2F Primer

SEQ ID NO: 6 Dw3/2R Primer

SEQ ID NO: 7 Dw3/3F Primer

SEQ ID NO: 8 Dw3/3R Primer

SEQ ID NO: 9 Dw3/4F Primer

SEQ ID NO: 10 Dw3/4R Primer

SEQ ID NO: 11 Dw3/5F Primer

SEQ ID NO: 12 dw3/IF_Neg Primer

SEQ ID NO: 13 dw3/IR_Neg Primer

### DETAILED DESCRIPTION

The dwarf phenotype (dw₁:Dw₂:dw₃:dw₄) of sorghum has been exploited since the 1940s. The dw₃ allele commonly used in commercial production is a 7765 base pair gene that contains a 882 base pair tandem duplication in exon 5 (positions 5650-6531) that disrupts the protein function. Karper noted that this dw₃ mutation produced a useful dwarf phenotype but was unstable and reverted to Dw3 tall (height) at a frequency of approximately 1 in 600 plants depending on the genetic background. Analyses of DNA sequences of dw₃ and revertant plants demonstrated that the instability of this allele was the result of unequal crossing-over that produced one wild-type allele and another allele with 3 tandem repeats.

The development of a stable dw₃I allele provides a useful genetic tool for addressing the problem of height mutants in sorghum. A PCR assay was developed to screen for new dw₃ alleles that are not capable of reverting to wild-type through unequal crossing-over (e.g. not based on the tandem duplication in exon 5).

### PCR Screening Assays

The Dw3 locus of the sorghum genome contains very high guanine-cytosine (GC) content (−73% GC). Hence, only very clean DNA and specially modified PCR reaction conditions can be used to successfully amplify the tandem duplication in exon 5 of the dw₃ allele.

Optimized PCR for dw₃ Using High-Quality DNA

Seeds of the African cultivar “El Mota” (Dw3/Dw3) and the USA parent lines “RTx430” dw₃/dw₃) and ‘BOK11” (dw₃/dw₃) were pretreated with fungicide and grown in plastic trays containing a soil mixture of ⅓ potting mix, ⅓ top soil and ⅓ sand in a greenhouse in Lilly Hall at Purdue University in West Lafayette, Ind. A tissue sample of the first immature leaf was collected at approximately 7 d after emergence. High quality DNA was extracted from these tissue samples representing the Dw3 and dw₃ alleles using a protocol modified from Richards et al. (2001), Vallejos (2007), and Clarke (2009). Fresh tissue was frozen using liquid nitrogen and pulverized using a mortar and a pestle. The tissue was transferred to 2 ml polypropylene collection tubes (Sigma Catalog Number: Z628034), approximately ⅓ of the tube was filled with homogenized tissue. The tissue was incubated with 600 μl of CTAB buffer (CTAB 2%, 100 mM TrisHCL pH 8.0, 50 mM EDTA, 1.4 M NaCL) containing 0.17% BME at 65° C. for 30 min. The samples were cleared of proteins and solid material using three organic solvent extractions starting with cholorform:octanol (24:1), followed by phenol:chloroform:octanol (25:24:1), followed by chloroform:octanol (24:1). After the final organic solvent extraction, the DNA was precipitated from the aqueous phase using an equal volume of cold isopropanol and 1/10 volume of 3M NaOAC pH 5.0 followed by centrifugation. The DNA pellet was resuspended in double distilled water and the DNA concentration was measured using a ThermoScientific NanoDrop™ 1000 Spectrophotometer. DNA from El Mota (Dw3/Dw3) was used as a check for the wild type Dw3 allele. DNA from RTx430 (dw₃/dw₃) and BOK11 (dw₃/dw₃) were used as checks for the mutant dw3 allele. Equal quantities of DNA isolated from RTx430 and El Mota were mixed to simulate a heterozygous individual (Dw3/dw3) or the genotype of expected Dw3-revertants.

The PCR reaction conditions for amplifying the target region of the dw₃ and Dw3 alleles were optimized by evaluating an array of PCR primer pairs, annealing temperatures, and PCR reaction adjuvants needed to melt the high GC content of this region. Different forward and reverse PCR primers flanking the duplicated region in exon 5 were designed with the expected PCR fragment sizes from dw₃ being approximately 1800 to 2300 bp and from Dw3 being approximately 900-1400 bp (Table 1). The primer set Dw3/1F and Dw3/1R were the same as those reported by Multani et al. (2003) (Seq. Nos. 12, 13). The other four primers were designed based on BTx623 sequence available online (Gen bank accession number EES 15161.1) and published by Paterson (2008) and Paterson et al. (2009) (Seq. Nos. 3-10). These forward and reverse primers were paired for PCR in an array of combinations and PCR was performed using the conditions described by Multani et al. (2003). Under these conditions, most forward and reverse primer pair combinations amplified the target region in Dw3 and dw3 dw₃. The smaller fragment from Dw3 (El Mota) was preferentially amplified when equal amounts of DNA from El Mota and RTx430 were mixed to simulate the heterozygous condition. Based on PCR yield, reproducibility, and preferential amplification of the smaller band, the primer set Dw3/4F-5R was selected for optimization.

The size of the PCR fragments from Dw3 and dw3 was 1077 and 1959 bp, respectively, when amplified using the primers Dw3/4F and Dw3/5R. A procedure modified from Lotte Hansen and Justeseni (2006) was used to evaluate efficacy of DMSO (5 to 10%), Glycerol (5 to 20%), or a combination of DMSO+Glycerol in different concentrations to improve PCR amplification of this high-GC amplicon. The combination of different concentrations of DMSO and glycerol did not significantly improve the PCR yield and reliability, but the addition of just 5% DMSO substantially enhanced amplification of the target fragment in Dw3 and dw₃ particularly the larger 1959 bp fragment dw₃. PCR reaction annealing temperature was optimized using a gradient PCR in a Bio-Rad C1000 thermal cycler. When 5% DMSO is added to the PCR reaction, the best annealing temperature for primer set Dw3 4F/5R was 67.5° C., which eliminated non-target amplification.

Based on these results, the optimal PCR conditions for amplifying the target site in exon 5 of dw₃ can be summarized as follows. First, only very clean DNA similar to the samples described above should be used in PCR reactions involving dw₃ because of the high GC-content of this gene. Several PCR primers were developed for this region but the best two primers identified to date are:

Forward primer Dw3/4F:
(SEQ ID NO: 9)
CGT CCT GCA GAA GAT GTT CAT GAA GG
Reverse primer Dw3/5R:
(Seq. No. 10)
GTG CGC CAC CAC GAT GGT GGT GC

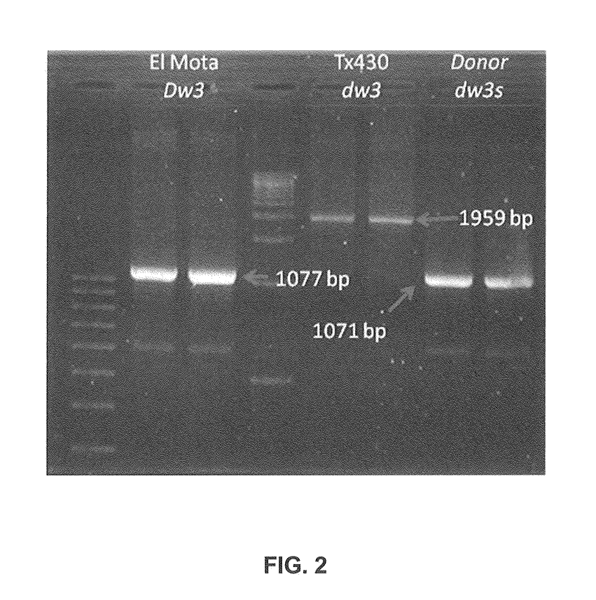

These primers should be used in PCR reactions that include 2 mM MgCl2, 0.4 mM dNTPs, 0.4 μM forward and reverse primers, 5% DMSO, and 0.5 units Promega GoTaq® DNA Polymerase in a 20 μl total reaction volume. The most optimal annealing temperature for primer set Dw3/4F and Dw3/5R was 67.5° C., higher than normal but not surprising given the high GC content of the target region. Under these conditions, the PCR reaction produces a 1959 bp product in dw₃ genotypes containing the tandem duplication and a 1077 bp product in Dw3 genotypes or genotypes without the tandem duplication (FIG. 2).

### High-Throughput DNA Extraction

A high-throughput DNA extraction protocol that produced high-quality DNA for amplification of the target site in dw₃ was needed for screening of large numbers of individual plants. Five different DNA extraction protocols were evaluated and modified to implement a high throughput screening protocol for use in sorghum seedlings. Plant tissue samples were taken from the first immature leaf of young seedlings representing El Mota (Dw3/Dw3) and the inbred lines RTx430 (dw₃/dw₃), BOK11 (dw₃/dw₃), and RTx2783 at approximately 7 d after emergence.

The first DNA extraction protocol was modified from the method described by Xin et al. (2003). A small leaf sample less than 0.2-0.5 cm was collected in a 96-well PCR reaction plate, sealed with adhesive film, reinforced with a compression pad to avoid sample evaporation (ABI prism catalog No 4312639), and incubated in 25 μl of Buffer A (100 mM NaOH, 2% Tween) at 95° C. for 15 min using a Bio-Rad C1000 thermal cycler, followed by 4° C. cycle for 30 min. For NaOH neutralization, 25 μl of Buffer B (100 mM Tris-HCL pH 8.0 and 2 mM EDTA) was added to the extract. Each sample was diluted five times and 4 μl of DNA extract was added directly to the PCR reaction. PCR amplification of the tandem duplication in dw₃ using the PCR conditions described herein produced poor and inconsistent amplification of the 1959 bp fragment from dw₃ and the 1077 bp fragment from Dw3.

The Sigma Extract-N-Amp Plant PCR Kit (Sigma Catalog Number XNAP2) was used next to determine suitability of this DNA extraction for amplification of the target sequence. This commercial procedure is similar to the protocol described by Xin et al. (2003), but differs in the addition of a solution to neutralize inhibitory substances. DNA extracted using this procedure was tested in PCR reactions to amplify the target sequence in dw₃ and Dw3. Evaluation of the PCR products in agarose gels indicated poor and inconsistent amplification of the 1959 bp fragment from dw₃ and the 1077 bp fragment from Dw3.

The third DNA extraction protocol evaluated in these experiments was adapted from Rinehart (2009). This “Salting Out DNA Extraction” procedure uses a micropestle to grind the tissue in DNA Extraction Buffer (Tris-HCL 100 mM, 50 mM EDTA, 2% SDS) followed by addition of ammonium acetate to separate the DNA from the debris and to precipitate proteins. The extract was centrifuged at 15,000 rpm for 10 min, the DNA was precipitated using isopropanol, and washed using 70% EtOH (ethanol). This protocol produced good quality DNA and could be used as template to amplify the 1077 bp product in Dw3, but it failed to consistently amplify the 1959 bp fragment in dw₃.

The last two protocols employ silica gel to dry plant tissue followed by mechanical disruption using a tissue homogenizer. The first step in both protocols involved collection of 2-3 cm leaf samples in 1.1 ml 8-TubeStrips containing dry silica gel, arranged in 8×12 microracks (ISC BioExpress Catalog number P-8705). The tissue was dried for 2-3 days at room temperature followed by grinding in a Troemner Homogenizer (Catalog No TR930146) at maximum speed or in a GenoGrinder 2000 (Spex CertiPrep, USA) at 1400 strokes min⁻¹ for 4 min. After tissue processing, the two protocols use different DNA extraction buffers and organic extraction reagents. The fourth DNA extraction protocol was modified from Mace et al. (2003) and Clarke (2009). The pulverized tissue was incubated in 400 μl of CTAB DNA Extraction Buffer (CTAB 1%, 100 mM Tris-HCL pH 8.0, 50 mM EDTA, 1.4 M NaCL) at 65° C. for 30 min. The solid material and polysaccharides were removed by extraction with 400 μl chloroform:octanol (24:1), followed by centrifugation for 30 min. The aqueous phase containing the DNA was transferred to a new microtube and the DNA was precipitated by addition of isopropanol and 1/10 volume of 3M NaOAC pH 5.0, followed by centrifugation for 30 min (3200 rpm). The DNA pellet was washed twice using 70% EtOH, air dried, and dissolved overnight in double distilled H2O (ddH2O). This protocol produced good quality DNA and could be used as template to amplify the 1077 bp product in Dw3, but it failed to consistently amplify the 1959 bp fragment in dw₃.

The last DNA extraction protocol evaluated in these experiments used an extraction buffer containing UREA (7M UREA, 0.3M NaCL, 50 mM Tris-HCL pH 8.0, 20 mM EDTA pH 8.0, 20% Sarcosine). Tissue samples were collected from plants, dried with silica gel, and pulverized in the Troemner Homogenizer or GenoGrinder 2000. These dried tissue samples were incubated in 400 μA of UREA buffer for 15 min at room temperature followed by organic extraction with phenol:chloroform:octanol (25:24:1) for 15 min. DNA was precipitated from the aqueous phase using isopropanol and 1/10 volume of 3M NaOAC pH 5.0, washed using 70% EtOH, and resuspended in 200 μl of ddH2O. PCR analysis of the dw₃ locus using this DNA demonstrated that it was suitable as template for amplification of the 1077 bp product in Dw3 AND the 1959 bp fragment in dw₃. Further optimization demonstrated that the procedure could be completed in approximately five hours and up to 384 samples could be processed per day. The average DNA extraction yields ranged from 20 to 40 μg of DNA, and the A260/A280 ratio was between 1.9 to 2.

Screening for de novo Mutant Alleles of dw₃

Multani et al. (2003) suggested that de novo mutant alleles of dw₃ might be generated by nucleotide substitutions produced during meiotic recombination and unequal crossing over. To test the feasibility of screening for these types of mutants, a high throughput DNA extraction procedure based on UREA buffer and a PCR genotyping procedure optimized for amplification of the tandem duplication in the dw₃ locus was used to screen for putative Dw3 revertants in three dw₃ sorghum inbred lines; RTx430 (Miller, 1984), RTx2783 (Peterson et at, 1984), and BOK11 (Johnson et al., 1982). The putative Dw3 revertants are plants that amplify the 1077 bp PCR product associated with the Dw3 allele.

A total of 3,864 seedlings of RTx430, RTx2783, and BOK11 were screened for the presence of the 1077 bp PCR product. The frequency of putative Dw3 revertants was high in RTx430 (8 out of 2328 seedlings) and lower in BOK11 (1 out of 1152 seedlings) and RTx2783 (0 out of 384 seedlings). The 1077 bp PCR product from each of the RTx430 revertants was purified and the DNA sequenced. Results from sequencing showed that the DNA sequence was the same as the sequence of wild type Dw3 allele in every case. This suggested that the unequal crossover event did not generate any deletions or de novo mutations in exon 5. To test this hypothesis, the putative revertant plants were transplanted to 5 L pots in the greenhouse to evaluate the expression of the plant height phenotype. Seven plants survived transplanting and exhibited a tall phenotype at maturity similar to dw₃. These plants were self-pollinated and the segregation analysis of the F2 generation confirmed a 3:1 segregation ratio for the effects of a single dominant gene.

### Identification of a Stable dw3 Allele in the Natural Gene Pool of Sorghum

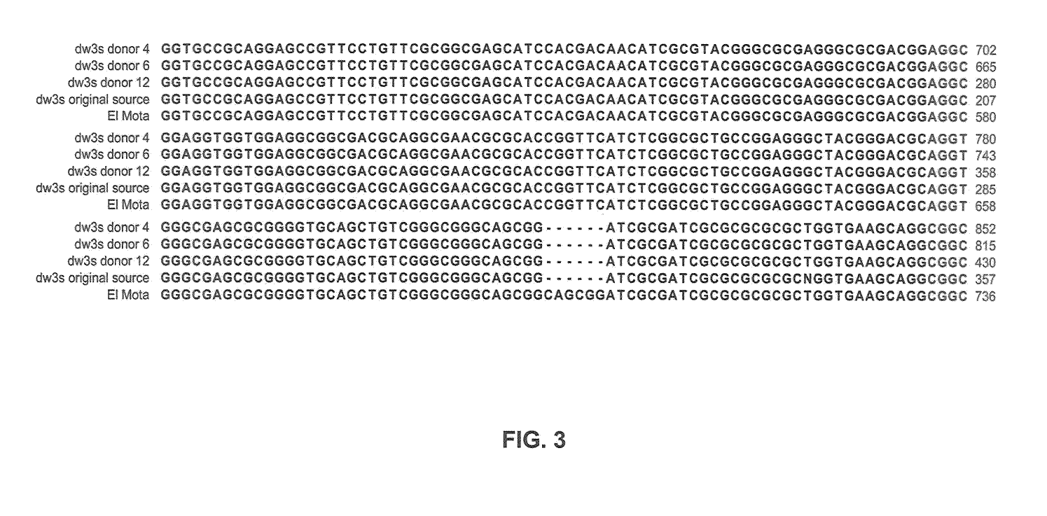

PCR analysis was used to screen sorghum genotypes used in commercial sorghum seed production to evaluate genetic variation for the tandem duplication represented in exon 5 of the dw₃ allele. The goal of these experiments was to identify dw₃ alleles that did not contain the duplication, but still produced a dw₃ phenotype because of some other mutation in the gene. DNA was extracted from diverse lines using a high throughput DNA extraction procedure based on UREA buffer and a PCR genotyping procedure optimized for amplification of the tandem duplication in the dw₃ locus. A natural variant of a % was found (FIG. 2). PCR analysis produced a 1071 bp product (FIG. 2). This PCR product was sequenced and the dwarf phenotype associated with this allele was found to be the result of a 6 base pair deletion in exon 5 (FIG. 3). This 6-bp deletion codes for amino acids Q1273 and R1274 of the ABC signature motif of the enzyme. This is a highly conserved domain and protein sequence comparisons of this motif in proteins from many different plant and animal species show essentially no genetic variation. Furthermore, since this mutation is a deletion and not a tandem duplication, the allele is not able to spontaneously revert to Dw₃, thereby providing a genetic solution to the problem of height mutants in sorghum. This allele was designated dw₃ₛ for “dw₃ stable”.

Although PCR and DNA sequencing demonstrated the existence of dw₃ₛ allele (FIGS. 2 and 3), it was critical to verify the stable dwarf phenotype of the line harboring it. To do this, approximately 5 lbs of seed was increased in a greenhouse at Purdue University. This seed lot was used to plant 0.07 ha and 0.10 ha plots in growout fields in Guayanilla, Puerto Rico. Visual observations of these plots were taken about 7 weeks after planting to quantify the frequency of spontaneous height mutants. Approximately 23,040 plants were evaluated in the first plot and no height mutants were observed. In the second plot, approximately 28,500 plants were evaluated and no height mutants were observed. These observations of zero height mutants in over 50,000 dw₃ₛ plants contrasts sharply with those reported by Karper (1932) who described a reversion frequency of approximately one mutant in every 600 plants having the unstable dw₃ allele. The stable dwarf phenotype of dw₃ₛ plants in field experiments confirmed the expectations from DNA sequencing that suggested that the 6 bp deletion in the dw₃ₛ allele should be completely stable.

The PCR primers described herein can be used to differentiate the stable dw3s allele from the unstable dw₃ allele. The stable allele produces a 1171 bp product and the unstable allele produces a 1951 bp product (FIG. 2). These markers provide an important tool in the development of stable dwarf inbreds and breeding populations because plants with the stable allele (dw₃ₛ) cannot be differentiated from plants with the unstable allele (dw₃) by visual inspection of individual plants. However, a quick screen of plant DNA using the PCR protocol described herein will indicate which plants carry the stable dw₃ₛ allele.

Table 1 lists PCRs primers evaluated for efficacy of amplification of the tandem duplication in dw₃ and Dw3. Primers flanking the duplication produce PCR fragments that range from approximately 900-bp to 1400-bp for Dw3 alleles and from 1800-bp to 2300-bp for the original dw₃ allele.

TABLE 1

| Forward | | Reverse | |
|---|---|---|---|
| Primer | Sequence | Primer | Sequence |
| Dw3/IF | CTCCTCGCCGT | Dw3/IR | GCGGCACCGCC |
| | CTTCCCGCTCG | | TGTCAGCCGCT |
| | SEQ ID NO: 3 | | SEQ ID NO: 4 |
| Dw3/2F | CGTTCAACGCGGA | Dw3/2R | CGCCTGTCAGC |
| | GCGCAAGATCACG | | CGCTGCAGCTG |
| | SEQ ID NO: 5 | | SEQ ID NO: 6 |
| Dw3/3F | GCTCTTCGAGG | Dw3/3R | CGTCGATCACG |
| | CCAACCTTCG | | GCGATGGTGTG |
| | SEQ ID NO: 7 | | SEQ ID NO: 8 |
| Dw3/4F | CGTCCTGCAGAAG | Dw3/5R | GTGCGCCACCAC |
| | ATGTTCATGAAGG | | GATGGTGGTGC |
| | SEQ ID NO: 9 | | SEQ ID NO: 10 |
| Dw3/5F | CGAGGCCGTC | dw3/ | GTTGTACTTGC |
| | GCCAACCTGC | IR_Neg | GCACGTCCTTG |
| | SEQ ID NO: 11 | | SEQ ID NO: 13 |
| dw3/ | GAGTCGGAGCG | | |
| IF_Neg | GTGGCTCTTC | | |
| | SEQ ID NO: 12 | | |

### PUBLICATIONS

The publications listed below are incorporated by reference to the extent they relate materials or methods disclosed herein.
Brown, P. J., W. L. Rooney, C. Franks, and S. Kresovich. 2008. Efficient Mapping of PH Quantitative Trait Loci in a Sorghum Association Population With Introgressed Dwarfing Genes. Genetics 180:629-637.
Clarke, J. D. 2009. Cetyltrimethyl Ammonium Bromide (CTAB) DNA Miniprep for Plant DNA Isolation. Cold Spring Harbor Protocols 2009:pdb.prot5177.
Karper, R. E. 1932. A Dominant Mutation of Frequent Recurrence in Sorghum. Am. Nat. 46: 511-529.
Mace, E., K. Buhariwalla, H. Buhariwalla, and J. Crouch. 2003. A high-throughput DNA extraction protocol for tropical molecular breeding programs. Plant Molecular Biology Reporter 21:459-460.
Miller, F. R. 1984. Registration of RTx430 Sorghum Parental Line. Crop Sci 24:1224.
Multani, D. S., S. P. Briggs, M. A. Chamberlin, J. J. Blakeslee, A. S. Murphy, and G. S. Johal. 2003. Loss of an MDR Transporter in Compact Stalks of Maize br2 and Sorghum dw3 Mutants. Science 302:81-84.
Paterson, A. H. 2008. Genomics of Sorghum. International Journal of Plant Genomics. 2008:1-6.
Paterson, A. H., J. E. Bowers, R. Bruggmann, I. Dubchak, J. Grimwood, H. Gundlach, G. Haberer, U. Hellsten, T. Mitros, A. Poliakov, J. Schmutz, M. Spannagl, H. Tang, X. Wang, T. Wicker, A. K. Bharti, J. Chapman, F. A. Feltus, U. Gowik, I. V. Grigoriev, E. Lyons, C. A. Maher, M. Martis, A. Narechania, R. P. Otillar, B. W. Penning, A. A. Salamov, Y. Wang, L. Zhang, N. C. Carpita, M. Reeling, A. R. Gingle, C. T. Hash, B. Keller, P. Klein, S. Kresovich, M. C. McCann, R. Ming, D. G. Peterson, R. Mehboob ur, D. Ware, P. Westhoff, K. F. X. Mayer, J. Messing, and D. S. Rokhsar. 2009. The Sorghum bicolor genome and the diversification of grasses. Nature 457:551-556.
Peterson, G. C., J. W. Johnson, G. L. Teetes, and D. T. Rosenow. 1984. Registration of Tx2783 Greenbug Resistant Sorghum Germplasm Line. Crop Sci 24:390.
Quinby, J. R. 1974. Sorghum Improvement and the Genetics of Growth. Texas A&M University Press, College Station, Tex.
Quinby, J. R. 1975. The Genetics of Sorghum Improvement. J Hered 66:56-62.
Richards, E., M. Reichardt, and S. Rogers. 2001. Current Protocols in Molecular Biology In F. M. Ausubel, et al., (eds.) Unit 2.3. Preparation of Genomic DNA from Plant Tissue, Vol. 2009. Wiley Interscience, New York.
Rinehart, K. D. 2009. Characterizing disease and pest resistance in wheat introgressed from related species. PhD Dissertation, Purdue University, West Lafayette.
Saghai-Maroof, M. A., K M Soliman, R A Jorgensen, and R W Allard. 1984. Ribosomal DNA spacer-length polymorphisms in barley: mendelian inheritance, chromosomal location, and population dynamics. Proceedings of the National Academy of Sciences 81:8014-8018.
US 2000/030816, Publication 01/034818 (Pioneer Hi Bred) (sequence of Dw₃)
Vallejos, C. E. 2007. An Expedient and Versatile Protocol for Extracting High-Quality DNA from Plant Leaves. Cold Spring Harbor Protocols 2007:pdb.prot4765.
Xin, Z. G., J. P. Velten, M. J. Oliver, and J. J. Burke. 2003. High-throughput DNA extraction method suitable for PCR. Biotechniques 34:820.

1. An isolated nucleic acid molecule comprising a fragment of the nucleotide sequence of the sorghum dw₃ gene, wherein the fragment comprises a deletion mutation in exon 5 of the dw₃ gene.

2. The isolated nucleic acid molecule of claim 1, wherein the deletion mutation is present in a region represented by nucleic acid position 259 to 264 of the exon 5.

3. The isolated nucleic acid molecule of claim 1, wherein the deletion mutation comprises absence of contiguous nucleic acids GCTGCC in exon 5 of the dw₃ gene.

4. An isolated nucleic acid molecule comprising the nucleotide sequence of the sorghum dw₃ gene, wherein the nucleic acid molecule comprises a deletion mutation in exon 5 of the dw₃ gene.

5. The isolated molecule of claim 4 designated dw₃ stable (dw₃ₛ)

6. The isolated nucleic acid molecule of claim 1, wherein the deletion mutation is present in a region represented by nucleic acid deletions at position 194 to 199 of SEQ ID NO: 1.

7. The isolated nucleic acid molecule of claim 1, wherein the fragment is amplified by a polymerase chain reaction with oligonucleotide primers having nucleotide sequences SEQ ID NO: 9 (forward) and SEQ ID NO: 10 (reverse).

8. An introgressed sorghum plant comprising the nucleic acid of claim 1, wherein the sorghum plant is a dwarf variety.

9. The introgressed sorghum plant of claim 8, wherein the plant is herbicide tolerant.

10. The introgressed sorghum plant of claim 8, wherein the plant is resistant to insects and/or pathogens.

11. The introgressed sorghum plant of claim 8, wherein the plant has been introgressed with a pollen parent comprising the dw3s mutant allele.

12. A hybrid sorghum plant comprising the nucleic acid of claim 1, wherein the sorghum plant is a dwarf variety.

13. The hybrid sorghum plant of claim 11 is introgressed.

14. A sorghum seed obtained from an introgressed sorghum plant comprising the nucleic acid of claim 1 or from a hybrid sorghum plant comprising the nucleic acid of claim 1.

15. A plurality of sorghum seeds obtained from introgressed sorghum plant comprising the nucleic acid of claim 1 or from a hybrid sorghum plant comprising the nucleic acid of claim 1.

16. The sorghum seeds of claim 15, wherein the resulting sorghum plants after germination are of uniform height.

17. The sorghum seeds of claim 15, wherein the resulting sorghum plants after germination do not display any height mutant.

18. The sorghum seeds of claim 15, wherein the resulting sorghum plants are genetically stable for the dw₃ₛ mutant allele.

19. A method of producing sorghum plants that have uniform plant height, the method comprising crossing a sorghum plant having the nucleic acid of claim 1 with a parental sorghum line to produce sorghum plants having uniform plant height.

20. The method of claim 19 further comprising introgressing the resulting sorghum plants.

21. The method of claim 19 further comprising introgressing the resulting sorghum plants with one or more hybrid parental lines.

22. A method of screening for the presence of a deletion mutant in exon 5 of the sorghum dw₃ gene, the method comprising detecting the presence of the deletion mutation in the exon 5 of the sorghum dw₃ gene by a polymerase chain reaction (PCR).

23. The method of claim 22, wherein the PCR is performed with oligonucleotide primers having nucleotide sequences SEQ ID NO: 9 (forward) and SEQ ID NO: 10 (reverse).

24. The method of claim 23, wherein the PCR results in an amplified product of length of about 1071 bp.

25. A method of screening for the presence of a deletion mutant in exon 5 of the sorghum dw₃ gene, the method comprising detecting the presence of the deletion mutation in the exon 5 of the sorghum dw₃ gene by a sequencing reaction.

26. A biomarker for determining the presence of a deletion mutant in exon 5 of the sorghum dw₃ gene, wherein the biomarker is an amplified fragment comprising the mutation in the exon 5 of the sorghum dw₃ gene.

27. The biomarker of claim 26, wherein the amplified fragment comprises a deletion of contiguous nucleic acids GCTGCC in exon 5 of the dw₃ gene.

28. An isolated sorghum plant comprising the nucleic acid of claim 1, wherein the sorghum plant is a dwarf variety.
